SEQUENCE LISTING (12 sequences)

SEQ ID NO: 1 - DNA, 18 bp, Candida albicans
aaagagcctgcgactatg

SEQ ID NO: 2 - DNA, 18 bp, Candida albicans
ggctgattcttcggtttg

SEQ ID NO: 3 - DNA, 20 bp, Candida albicans
ctacttccacagctgcttcc

SEQ ID NO: 4 - DNA, 20 bp, Candida albicans
gttgaagttgcagatggagc

SEQ ID NO: 5 - DNA, 20 bp, Candida albicans
cctcctgtagtatttctccc

SEQ ID NO: 6 - DNA, 20 bp, Candida albicans
ggagggaagagatggtaatc

SEQ ID NO: 7 - DNA, 20 bp, Candida albicans
catcaataccacccacaagc

SEQ ID NO: 8 - DNA, 20 bp, Candida albicans
cacctactggaatcatcctc

SEQ ID NO: 9 - DNA, 20 bp, Candida albicans
ggtagctggatctacagttc

SEQ ID NO: 10 - DNA, 18 bp, Candida albicans
gaacaacgtatctccacg

SEQ ID NO: 11 - DNA, 18 bp, Candida albicans
cagtgaacggaagctaag

SEQ ID NO: 12 - DNA, 21 bp, Candida albicans
ctcttgattaacttggccagg

(19) United States
(12) Patent Application Publication
(10) Pub. No.: US 2015/0176084 A1
(43) Pub. Date: Jun. 25, 2015

(54) CANDIDA ALBICANS OLIGONUCLEOTIDES, DETECTION METHOD, AND KIT THEREOF

(71) Applicant: Instituto Potosino de Investigación Cientifica y Tecnológica A.C., San Luis Potosi (MX)

(72) Inventors: Irene Beatriz CASTAÑO NAVARRO, San Luis Potosi (MX); Alejandro DE LAS PEÑAS NAVA, San Luis Potosi (MX)

(21) Appl. No.: 14/519,814

(22) Filed: Oct. 21, 2014

Related U.S. Application Data
(60) Provisional application No. 61/894,974, filed on Oct. 24, 2013.

(51) Int. Cl. C12Q 1/68

(52) U.S. Cl. CPC C12Q 1/6895; C12Q 2600/16; C12Q 2600/158

(57) ABSTRACT

The invention discloses an in vitro method for the identification of Candida albicans, the sequences associated to said identification, as well as diagnosis kits for identifying Candida albicans.

### CROSS-REFERENCE TO RELATED APPLICATIONS

This application claims the benefit of priority to U.S. Provisional Application No. 61/894,974 filed Oct. 24, 2013, the contents of which are incorporated herein by reference.

### TECHNICAL FIELD OF THE INVENTION

The present invention belongs to the biotechnology field, especially to methods for detecting infectious diseases.

### BACKGROUND OF THE INVENTION

The incidence of hospital infections by opportunistic fungal pathogens has increased substantially in the last two decades, especially among patients immuno-suppressed or serious underlying diseases. Candidas are the most common fungal pathogens affecting humans. Several epidemiologic studies around the world report that the invasive infections with Candidas have increased. Therefore, for example the Center for Disease Control and Prevention (CDC) is requiring sensitive, specific and rapid detection and identification methods for this kind of fungi.

Although more than 100 Candida species are known, only four are responsible for about 95% hematological infections and 95-97% of invasive infections caused by Candida in US hospitals.

In the case of hematological infections the most frequent species are: Candida albicans (45.6%), Candida glabrata (26%), Candida parapsilosis (15.7%) and Candida tropicalis (8.1%). These proportions vary depending on the patient's condition, but are the same four species causing 95% of overall candidiasis.

Current detection methods are imprecise and take several days for determining the kind of Candida in biological samples. This provokes that the patient's treatment is inadequate and the mortality in hospitals is increased as well as health care costs. Molecular detection methods based on ITS or rDNA sequences usually have a high incidence of false positive or negative results because of the close phylogenetic relation among the different Candida species. Also, further analysis is required, since the ITS or rDNA sequences are of similar size and should be re-sequenced before a final result is provided. Examples of these kind of inventions are disclosed in EP2235214B1, AU2014202147A1, CN103740834A, US2013309683A1, which are incorporated only as reference and should not be considered as prior art for the instant invention.

Additionally, molecular methods detecting several Candida species have been designed, wherein there is a common oligonucleotide and an additional oligonucleotide specie specific, however these methods also need further analysis in order to differentiate each specie. Examples of such inventions are disclosed in CA2407226C, EP1960536B1, EP2315853B1, US8501408B2, US2013034856A1, which are incorporated only as reference and should not be considered as prior art for the instant invention.

Buchman et al. were the first to describe the use of PCR for identification of C. albicans in clinical samples (Buchman et al., Surgery 108:338-47 (1990)). These researchers used PCR to amplify part of a specific gene encoding cytochrome lanosterol 14-alpha demethylase. The predicted PCR product was approximately 240 bp, however unexplained amplification patterns were observed in several clinical samples containing DNA from C. albicans. In addition, the set of primers used by Buchman et al. amplified DNA from species other than C. albicans, resulting in PCR products with the ‘predicted’ size of 240 bp.

It has been reported that several Candida species have chromosome rearrangements that may cause loss of genetic material. (Butler, G., et al, Nature 459(7247):657-662 (2009)). This can be associated with variations in molecular diagnosis, since the target sequence may vary or lost.

In light of the above, the present invention discloses an in vitro method for detecting and identifying Candida albicans, with at least one specific oligonucleotide, but also with an in-block multiplex set of specific oligonucleotides, which allows identification of Candida albicans in clinical samples of different population subgroups.

### SUMMARY OF THE INVENTION

The present invention claims and discloses oligonucleotides for the specific identification of Candida albicans, consisting of a nucleic acid having at least 90% sequence homology to one of SEQ ID NOS: 1 to 12 or a complement thereof.

In a further embodiment, it is further disclosed an in vitro method for the specific identification of C. albicans, comprising the steps of: a) amplifying DNA fragments from a biological sample with at least one oligonucleotide as above defined; and b) identify the amplified DNA fragments; wherein in an specific embodiment the amplification of DNA fragments is carried out with at least one pair of oligonucleotides or at least two pair of oligonucleotides.

In an additional embodiment, a kit for the specific identification of Candida albicans, comprising at least one oligonucleotide as above mentioned is also disclosed; wherein in an specific embodiment, said kit comprises at least one oligonucleotide pair or at least two pair of oligonucleotides.

### BRIEF DESCRIPTION OF THE DRAWINGS

FIG. 1. shows a 2% agarose gel showing ribosomal DNA amplification of multiple Candida species by using the universal oligonucleotides ITS1 and ITS4. C. glabrata was used as positive control (BG14). For the electrophoresis, it was employed ⅕ of the total volume (2 μL) of the PCR amplification product of all of the samples and products. Lane 1 shows the molecular weight marker (1 Kb DNA Ladder Invitrogene); Lane 2 shows the Positive control C. glabrata; Lane 3 shows the negative control without DNA; Lane 4 shows C. glabrata; Lane 5 shows C. albicans; Lane 6 shows C. tropicalis, Lane 7 shows C. parapsilosis; Lane 8 shows C. bracarensis 1; Lane 9 shows C. bracarensis 2; Lane 10 shows C. bracarensis 3; Lane 11 shows C. bracarensis 4; Lane 12 shows C. bracarensis 5; lane 13 shows C. bracarensis 6; Lane 14 shows C. bracarensis 7; Lane 15 shows C. dubliniensis 1; Lane 16 shows C. dubliniensis 2; Lane 17 shows C. guillermondii; Lane 18 shows C. krusei 1; Lane 19 shows C. krusei 2 and Lane 20 shows the molecular weight marker.

FIG. 2 shows a 2% agarose gel showing temperature gradient for the C. albicans detection (SC5314) using oligonucleotide pair Ca2. The unspecific bands for C. glabrata, C. parapsilosis and C. dubliniensis disappear as the oligonucleotides annealing temperature increases, for this oligonucleotides pair, the optimal temperature is 67.7° C. For electrophoresis, the samples were run at a concentration 4 times higher than the one used for the controls. The amplification band for C. albicans has a length of 202 bp. Lane 1 shows the molecular weight marker (1 Kb DNA Ladder Invitrogene); Lanes 2-7 show Annealing temperature 61° C.: Lane 2 shows positive control C. albicans; Lane 3 shows negative control without DNA; Lane 4 shows C. glabrata; Lane 5 shows C. parapsilosis; Lane 6 shows C. dubliniensis. Lanes 7-11 show annealing temperature 61.7° C.: Lane 7 show the positive control C. albicans; Lane 8 shows the negative control without DNA; Lane 9 shows C. glabrata; Lane 10 shows C. parapsilosis; Lane 11 shows C. dubliniensis. Lanes 12-16 show annealing temperature 62.6° C.: Lane 12 shows the positive control C. albicans; Lane 13 shows the negative control without DNA; Lane 14 shows C. glabrata; Lane 15 shows C. parapsilosis; Lane 16 shows C. dubliniensis. Lane 17 shows the molecular weight marker. Lane 18 shows the molecular weight marker. Lanes 19-23 show annealing temperature 63.8° C.: Lane 19 shows the positive control C. albicans; Lane 20 shows the negative control without DNA; Lane 21 shows C. glabrata; Lane 22 shows C. parapsilosis; Lane 23 shows C. dubliniensis. Lanes 24-28 show annealing temperature 65.4° C.: Lane 24 shows the positive control C. albicans; Lane 25 shows the negative control without DNA; Lane 26 shows C. glabrata; Lane 27 shows C. parapsilosis; Lane 28 shows C. dubliniensis. Lanes 29-33 show annealing temperature 66.7° C.: Lane 29 shows the positive control C. albicans; Lane 30 shows the negative control without DNA; 11: C. glabrata; Lane 32 shows C. parapsilosis; Lane 33 shows C. dubliniensis. Lane 34 shows the molecular weight marker. Lane 35 shows the molecular weight marker. Lanes 36-40 show annealing temperature 67.6° C.: Lane 36 shows the positive control C. albicans; Lane 37 shows the negative control without DNA; Lane 38 shows C. glabrata; Lane 39 shows C. parapsilosis; Lane 40 shows C. dubliniensis. Lanes 41-45 show annealing temperature 68° C.: Lane 41 shows the positive control C. albicans; Lane 42 shows the negative control without DNA; Lane 43 shows C. glabrata; Lane 44 shows C. parapsilosis; Lane 45 shows C. dubliniensis. Lane 46 shows the molecular weight marker.

FIG. 3. shows a 2% agarose gel showing temperature gradient for C. albicans detection (SC5314) using the oligonucleotides pair Ca5. The unspecific strip for C. guilliermondii disappears as the oligonucleotides annealing temperature increases, for this oligonucleotides pair, the optimal temperature selected is 63.5° C. Lane 1 shows the molecular weight marker (1 Kb DNA Ladder Invitrogene). Lanes 2-4 show the annealing temperature 56° C. Lane 2 shows the positive control C. albicans; Lane 3 shows the negative control without DNA; Lane 4 shows C. guillermondii. Lanes 5-7 show annealing temperature 57.1° C. Line 5 shows the positive control C. albicans; Lane 6 shows the negative control without DNA; Lane 7 shows C. guillermondii. Lanes 8-10 show annealing temperature 58.8° C. Lane 8 shows the positive control C. albicans; Lane 9 shows the negative control without DNA; Lane 10 shows C. guillermondii. Lanes 11-13 show annealing temperature 60.8° C. Lane 11 shows the positive control C. albicans; Lane 12 shows the negative control without DNA; Lane 13 shows C. guillermondii. Lanes 14-16 show annealing temperature 63.5° C. Lane 14 show the positive control C. albicans; Lane 15 shows the negative control without DNA; Lane 16 shows C. guillermondii. Lanes 17-19 show annealing temperature 65.7° C. Lane 17 show the positive control C. albicans; Lane 18 shows the negative control without DNA; Lane 19 shows C. guillermondii. Lanes 20-21 show the molecular weight marker. Lanes 22-24 show the annealing temperature 67.2° C. Lane 22 shows the positive control C. albicans; Lane 23 shows the negative control without DNA; Lane 24 shows C. guillermondii. Lanes 25-27, show annealing temperature 68° C. Lane 25 shows the positive control C. albicans; Lane 26 shows the negative control without DNA; Lane 27 shows C. guillermondii.

FIG. 4. shows a 2% agarose gel showing temperature gradient for C. albicans detection (SC5314) using the oligonucleotides pair Ca6. For this oligonucleotides pair, the optimal temperature selected is 60.8° C. For electrophoresis, the samples were run at a concentration 4 times higher than the one used for the controls. Lane 1 shows the molecular weight marker (1 Kb DNA Ladder Invitrogene). Lanes 2-4 show annealing temperature 56° C. Lane 2 shows the positive control C. albicans; Lane 3 shows the negative control without DNA; Lane 4 shows C. guillermondii. Lanes 5-7 show annealing temperature 57.1° C. Lane 5 shows the positive control C. albicans; Lane 6 shows the negative control without DNA; Lane 7 shows C. guillermondii. Lanes 8-10 show annealing temperature 58.8° C. Lane 8 shows the positive control C. albicans; Lane 9 shows the negative control without DNA; Lane 10 shows C. guillermondii. Lanes 11-13 show annealing temperature 60.8° C. Lane 11 shows the positive control C. albicans; Lane 12 shows the negative control without DNA; Lane 13 show C. guillermondii. Lanes 14-16 show annealing temperature 63.5° C. Lane 14 shows the positive control C. albicans; Lane 15 show the negative control without DNA; Lane 16 shows C. guillermondii. Lanes 17-19 show annealing temperature 65.7° C. Lane 17 shows the positive control C. albicans; Lane 18 shows the negative control without DNA; Lane 19 shows C. guillermondii. Lanes 20-21 show the molecular weight marker. Lanes 22-24 show the annealing temperature 67.2° C. Lane 22 shows the positive control C. albicans; Lane 23 shows the negative control without DNA; Lane 24 shows C. guillermondii. Lanes 25-27 show annealing temperature 68° C. Lane 25 shows the positive control C. albicans; Lane 26 shows the negative control without DNA; Lane 27 shows C. guillermondii.

FIGS. 5 A-H. show a 2% agarose gel showing oligonucleotide concentration analysis for C. albicans detection (SC5314) using the oligonucleotides pair Ca2. For this oligonucleotides pair, the optimal concentration selected is 200 nM. For electrophoresis, the samples were run at a concentration 4 times higher than the one used for the controls;

FIG. 5 A: shows an oligonucleotides pair concentration of 100 nM;

FIG. 5 B shows an oligonucleotides pair concentration of 200 nM;

FIG. 5 C shows an oligonucleotides pair concentration of 400 nM;

FIG. 5 D shows an oligonucleotides pair concentration of 500 nM;

FIG. 5 E shows an oligonucleotides pair concentration of 600 nM;

FIG. 5 F shows an oligonucleotides pair concentration of 800 nM;

FIG. 5 G shows an oligonucleotides pair concentration of 1000 nM;

FIG. 5 H shows an oligonucleotides pair concentration of 1200 nM. For each gel, the lane order is: Lane 1 shows the molecular weight marker (1 Kb DNA Ladder Invitrogene); Lane 2 shows the positive control C. albicans; Lane 3 shows the negative control without DNA; Lane 4 shows C. glabrata; Lane 5 shows C. tropicalis; Lane 6 shows C. parapsilosis; Lane 7 shows C. dubliniensis; Lane 8 shows C. bracarensis; Lane 9 shows C. guillermondii; Lane 10 shows C. krusei.

FIGS. 6A-6H. shows a 2% agarose gel showing oligonucleotide concentration analysis for C. albicans detection (SC5314) using the oligonucleotides pair Ca5. For this oligonucleotides pair, the optimal concentration selected is 200 nM. For electrophoresis, the samples were run at a concentration 4 times higher than the one used for the controls;

FIG. 6 A shows an oligonucleotides pair concentration of: 100 nM;

FIG. 6 B shows an oligonucleotides pair concentration of 200 nM;

FIG. 6 C shows an oligonucleotides pair concentration of 400 nM;

FIG. 6 D shows an oligonucleotides pair concentration of 500 nM;

FIG. 6 E shows an oligonucleotides pair concentration of 600 nM;

FIG. 6 F shows an oligonucleotides pair concentration of 800 nM;

FIG. 6 G shows an oligonucleotides pair concentration of 1000 nM;

FIG. 6 H shows an oligonucleotides pair concentration of 1200 nM. For each gel, the lane order is: 1: molecular weight marker (1 Kb DNA Ladder Invitrogene); Lane 2 positive control C. albicans; 3: negative control without DNA; 4 C. glabrata; 5: C. tropicalis; 6: C. parapsilosis; 7: C. dubliniensis; 8: C. bracarensis; 9: C. guillermondii; 10: C. krusei.

FIGS. 7A-7I. show a 2% agarose gel showing oligonucleotide concentration analysis for C. albicans detection (SC5314) using the oligonucleotides pair Ca6. For this oligonucleotides pair, the optimal concentration selected is 300 nM.

FIG. 7 A shows an oligonucleotides pair concentration of: 100 nM;

FIG. 7B shows an oligonucleotides pair concentration of 200 nM;

FIG. 7C shows an oligonucleotides pair concentration of 300 nM;

FIG. 7D shows an oligonucleotides pair concentration of 400 nM;

FIG. 7E shows an oligonucleotides pair concentration of 500 nM;

FIG. 7F shows an oligonucleotides pair concentration of 600 nM;

FIG. 7G shows an oligonucleotides pair concentration of 800 nM;

FIG. 7H shows an oligonucleotides pair concentration of 1000 nM;

FIG. 7I shows an oligonucleotides pair concentration of 1200 nM. For each panel, the lane order is: Lane 1 shows the molecular weight marker (1 Kb DNA Ladder Invitrogene); Lane 2 shows the positive control C. albicans; Lane 3 shows the negative control without DNA; Lane 4 shows C. glabrata; Lane 5 shows C. tropicalis; Lane 6 shows C. parapsilosis; Lane 7 shows C. dubliniensis; Lane 8 shows C. bracarensis; Lane 9 shows C. guillermondii; Lane 10 shows C. krusei.

FIGS. 8A-8C. show a 2% agarose gel showing the analysis of the 36 clinical isolated samples C. albicans detection (SC5314) using the oligonucleotides pair Ca2. There were 12 samples detected as positive. In FIGS. 8 A-8C, lane 1 and 16 show the molecular weight marker (1 Kb DNA Ladder Invitrogene). In FIGS. 8A-8C lane 2 shows the positive control C. albicans. In FIGS. 8A-8C lane 3 shows the negative controls, without DNA. In FIGS. 8A-8C lanes 4 to 15 show the clinical samples.

FIGS. 9A-9B show a 2% agarose gel showing the analysis of the 36 clinical isolated samples for C. albicans detection (SC5314) using the oligonucleotides pair Ca5. There were 12 samples detected as positive;

In FIGS. 9A and 9B Lane 1 shows the molecular weight marker (1 Kb DNA Ladder Invitrogene) Lane 2 shows the positive control C. albicans; and Lane 3 shows the negative controls, without DNA. The remaining lanes 4 to 20 show clinical samples.

FIGS. 10A-10b show a 2% agarose gel showing the analysis of the 36 clinical isolated samples for C. albicans detection (SC5314) using the oligonucleotides pair Ca6. There were 11 samples detected as positive; the isolated sample AN17 was not detected as positive with Ca6, but it was positive for Ca2 and Ca5;

In FIGS. 10A-10b Lane 1 shows the molecular weight marker (1 Kb DNA Ladder Invitrogene); Lane 2 shows the positive control C. albicans; and Lane 3 shows the negative controls, without DNA. Remaining lanes 4 to 20 show clinical samples.

FIG. 11. shows a 2% agarose gel showing a multiplex test for C. albicans. Ca2, Ca5 and Ca6 oligonucleotide pairs were tested in several conditions. Predicted amplification sizes 173, 202 and 203 base pairs were detected in samples containing only C. albicans. Lane 1 shows the Molecular weight marker (1 Kb DNA Ladder Invitrogene). Lane 2 shows the C. albicans, C. glabrata, C. tropicalis, C. parapsilosis, C. dubliniensis, S. cerevisiae, 100 ng each. Lane 3 shows the negative control containing C. glabrata, C. tropicalis, C. parapsilosis, C. dubliniensis, S. cerevisiae, 100 ng each. Lane 4 shows the molecular weight marker. Lane 5 shows C. albicans. Lane 6 shows without DNA. Lane 7 shows C. albicans. Lane 8 shows C. glabrata. Lane 9 shows C. tropicalis. Lane 10 shows C. parapsilosis. Lane 11 shows C. dubliniensis.

FIG. 12A show 2% agarose gel showing specificity test with Ca2 oligonucleotide pair. In FIG. 12A Lane 1 shows the molecular weight marker (1 Kb DNA Ladder Invitrogene), Lane 2 shows the positive control C. albicans, Lane 3 shows the negative control without DNA, and Lane 4 shows C. albicans 100 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 5 shows C. albicans 10 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 6 shows C. albicans 1 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 7 shows 50 ng C. tropicalis, C. parapsilosis, C. glabrata, C. dubliniensis, C. bracarensis, C. guilliermondii, C. krusei, C. metapsilosis, C. orthopsilosis, S. cerevisiae each.

FIG. 12B shows Ca5 oligonucleotide pair. In FIG. 12B, Lane 1 shows molecular weight marker (1 Kb DNA Ladder Invitrogene), Lane 2 shows the positive control C. albicans, Lane 3 shows the negative control without DNA, Lane 4 shows C. albicans 100 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 5 shows C. albicans 10 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 6 shows C. albicans 1 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 7 shows 50 ng C. tropicalis, C. parapsilosis, C. glabrata, C. dubliniensis, C. bracarensis, C. guilliermondii, C. krusei, C. metapsilosis, C. orthopsilosis, S. cerevisiae each.

FIG. 12C shows Ca6 oligonucleotide pair. In FIG. 12C Lane 1 shows molecular weight marker (1 Kb DNA Ladder Invitrogene), Lane 2 shows the positive control C. albicans, Lane 3 shows the negative control without DNA, and Lane 4 shows C. albicans 100 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 5 shows C. albicans 10 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 6 shows C. albicans 1 ng plus 50 ng C. tropicalis, C. parapsilosis, C. glabrata, C. dubliniensis, C. bracarensis, C. guilliermondii, C. krusei, C. metapsilosis, C. orthopsilosis, S. cerevisiae each. Lane 7 shows 50 ng C. tropicalis, C. parapsilosis, C. glabrata, C. dubliniensis, C. bracarensis, C. guilliermondii, C. krusei, C. metapsilosis, C. orthopsilosis, S. cerevisiae each.

FIG. 13. shows Real-Time PCR example with Ca2 oligonucleotide pair. Panel A: Resulting curve of PCR after 40 cycles. X-axis: number of cycles; Y-axis ΔRn. Panel B: copy number result. X-axis: quantity; Y-axis: Cr.

### DETAILED DESCRIPTION OF THE INVENTION

The present invention discloses an in vitro method for detecting and identifying Candida albicans, with at least one set of specific oligonucleotides, but also with an in-block multiplex set of specific oligonucleotides, which allows identification of Candida albicans in clinical samples of different population subgroups with 100% of specificity and sensitivity.

Several oligonucleotides have been designed in order to specifically detect different chromosomal sites of Candida albicans. The amplified sequences are located in several chromosomes and in contigs that have unique regions that allow said specific detection. The different sizes among the amplification products of each pair of oligonucleotides allow that they are rapidly recognized in separate or a single multiplex assay. Candida albicans can be specifically detected by any amplification method, such as PCR, RT-PCR, Q-PCR, Southern blot, Dot blot, multiplex-PCR, nested-PCR, or any other amplification or nucleic acid detection methods.

“Amplification” should be interpreted as a process for artificial increasing the number of copies of a particular nucleic acid fragments into millions of copies through the replication of the target segment.

By “complementary” is meant a contiguous sequence that is capable of hybridizing to another sequence by hydrogen bonding between a series of complementary bases, which may be complementary at each position in the sequence by standard base pairing (e.g., G:C, A:T or A:U pairing) or may contain one or more positions, including a basic ones, which are not complementary bases by standard hydrogen bonding. Contiguous bases are at least 80%, preferably at least 90%, and more preferably about 100% complementary to a sequence to which an oligomer is intended to specifically hybridize. Sequences that are “sufficiently complementary” allow stable hybridization of a nucleic acid oligomer to its target sequence under the selected hybridization conditions, even if the sequences are not completely complementary.

“Sample preparation” refers to any steps or methods that prepare a sample for subsequent amplification and detection of Candida nucleic acids present in the sample. Sample preparation may include any known method of concentrating components from a larger sample volume or from a substantially aqueous mixture, e.g., any biological sample that includes nucleic acids. Sample preparation may include lysis of cellular components and removal of debris, e.g., by filtration or centrifugation, and may include use of nucleic acid oligomers to selectively capture the target nucleic acid from other sample components.

The present invention discloses several oligonucleotides for the specific identification of C. albicans, wherein said oligonucleotides comprises a continuous sequence of about 18 to 21 nucleotides of a target sequence. Said target sequence is located along the chromosomes of said C. albicans, in exclusive sites that allows non-cross reactions with any other kind of organism, including other Candida species and microbial or eukaryotic nucleic acid that can be contained in a biological sample.

Also, the oligonucleotides for the specific identification of Candida albicans, consist of a nucleic acid having at least 90% sequence homology to one of SEQ ID NOS: 1 to 12 or complements thereof.

Said oligonucleotides are sufficiently complementary to the target sequences of C. albicans. For the experimental procedures, the amplified sequences were re-sequenced in order to make sure that the amplified product corresponds to the disclosed genomic region.

This invention also discloses an in vitro method for the specific identification of C. albicans, comprising the steps of: a) amplifying nucleic acid fragments from a biological sample by an amplification method with at least one of the specifically designed oligonucleotides, such as those disclosed on SEQ ID NOS: 1 to 12 or a complement thereof; and b) identify the amplified nucleic acid fragments. In this method the biological sample is derived from one subject to study. The subject to study is a mammal, wherein as a preferred embodiment, but not limited, is a human. Additionally, in a preferred embodiment, said biological sample is selected from the group consisting of any sample containing DNA, fluids, tissue, or cell debris, midstream urine, urine culture tube, growing by nephrostomy (right and left kidney), water, hemodialysis, pleural fluid, culture pyogenic, mieloculture, bone marrow, blood lysis (peripheral blood), blood culture (blood), leukocyte concentrate, concentrated red cell, throat, nasal discharge, vaginal discharge, exudate prostate sputum, catheter, biopsies from different tissues such as lymph node, subcutaneous tissue, cornea, lung, pulmonary nodule, pancreas, jaw, skin, skin quantitative (cellulite, breast, scrotum, arm, hand), hair, nails, warm muscle, bone, breast, synovial fluid, scar, thigh, joint capsule, knee, omentum, bronchoalveolar lavage (lingula, upper and lower lobe (left and right), left and right LBA (airways)); post-mortem (liver, lung, spleen), wound swabs (perianal, vaginal, ulcer (foot, hand)), abscess (thigh, kidney, perianal) or peripancreatic.

Furthermore, a kit for the specific identification of Candida albicans, with at least one oligonucleotide or as a multiplex identification kit is disclosed. Said kits comprise at least one oligonucleotide specifically designed for the identification of Candida albicans such as those disclosed on SEQ ID NOS: 1 to 12 or complements thereof. In the multiplex embodiment, the kit comprises at least one oligonucleotide pair or more preferably, at least two oligonucleotide pairs.

The use of said oligonucleotides specifically designed for the specific identification of Candida albicans, is disclosed as well.

As an additional embodiment, the present invention discloses at least one probe useful for the specific identification of Candida albicans. Said identification is carried out by an in vitro method comprising coupling nucleic acid fragments from a biological sample with said probes and identifying the hybridized nucleic acid fragments, wherein said steps are carried out by any hybridization method.

In order to test fully the competitive advantage of the methods of the present invention against traditional diagnostic methods, below is a comparison of the times of two tests:

Traditional method of identification of Candida in urine, urine samples are analyzed in an automated urine analyzer coupled Urisys type UF-IOOi. The analysis was performed by flow cytometry with an argon laser. The UF-IOOi measures the properties of scattered light and fluorescence to count and identify the particles in the urine. The volume of the particles is determined from the impedance signals. Thus, according to the scatterplots, the result indicates which urine samples are likely to contain yeast cells. These samples are marked as YLC urine samples (yeast cells). In urine samples taken YLC marked 1 μl and plating medium Sabourand/Dextrose (SDA) and medium Sabourand/Dextrose with cefoperazone (CFP). These plates are incubated at 30° C. for 72 hours. Urine cultures with growth less than 10,000 CFU/ml, as no growth plates, are reported as not developed fungi (negative) urine cultures with equal or greater development to 10,000 CFU/ml pass germ tube test, with incubation for 2 hours at 35° C. In the case of negative germ tube is reported as Candida sp. To identify the species from the report of Candida sp. Vitek cards are used that allow the identification by means of assimilation of carbohydrates. These cards are incubated for a period of 24 to 48 hours, at which time the cards are read. The minimum total time to identify C. albicans, is 6 days, with a sensitivity of about 85%.

In the method for identifying C. albicans, of the present invention, the urine samples are analyzed in an automated urine analyzer coupled Urisys type UF-IOOi. The analysis was performed by flow cytometry with an argon laser.

The UF-IOOi measures the properties of scattered light and fluorescence to count and identify the particles in the urine. The volume of the particles is determined from the impedance signals. Thus, according to the scatterplots, the result indicates which urine samples are likely to contain yeast cells. These samples are marked as YLC urine samples (yeast cells). The time of this first stage is 2 hours. Next, in urine samples taken YLC marked as 1 ml, centrifuged, the supernatant is discarded, resuspended and boiled the pill. The genomic DNA obtained is used for PCR analysis using primers generated from the SEQ ID Nos. 1 to 12, under optimal conditions reaction. The PCR products were separated by agarose gel electrophoresis and the products are analyzed for the correct identification of C. albicans, together as an inblock multiplex or separately. The total test time is 6 hours.

Traditional method of identification of Candida in blood samples: Blood samples are incubated for 72 hours in the automated equipment BACTEC9240. When no growth of microorganisms metabolize these nutrients in the culture medium by releasing CO2. The release of CO2 is detected by the computer and automatically marked as blood cultures positive for yeast. Positive blood cultures for yeasts are grown on plates Sabourand/Dextrose (SDA) and Sabourand/Dextrose with cefoperazone (CFP) and incubated at 30° C. for 72 hours. Blood cultures with lower growth of 10,000 CFU/ml as well as those without growth, are reported as not develop fungus (negative), the blood cultures with growth equal to or greater than 10,000 CFU/ml was performed germ tube test for 2 hours at 35° C. In the case of negative germ tube is reported as Candida sp. To identify the species from the report of Candida sp. Vitek cards are used that allow the identification by means of assimilation of carbohydrates. These cards are incubated for 24 to 48 hours and are read to identify C. albicans. The total time for identification is a minimum of 9 days.

Method to detect C. albicans, according to the present invention in blood samples: Blood samples are incubated for 72 hours BACTEC9240 automated equipment. When no growth of microorganisms metabolize these nutrients in the culture medium by releasing CO2. The release of CO2 is detected by the computer and automatically marked as blood cultures positive for yeast. Blood samples positive for yeast marked 100 μl taken, centrifuged, the supernatant is discarded, resuspended and boiled the pill. The genomic DNA obtained from PCR annealing is used where any of the oligonucleotides generated from SEQ ID Nos. 1 to 12, in optimal reaction conditions. The PCR products were separated by agarose gel electrophoresis and the products are analyzed for the correct identification of C. albicans. The total test time is 3 days.

An alternate method is to take as the patient's blood sample without being seeded by blood culture. In this case, follow the above procedure and the total test time is 4 hours.

Thus, the critical step is to obtain sufficient genomic DNA from any of the types of samples described above, and from them, using genomic DNA obtained as the PCR template, and using any one of the oligonucleotides generable or generated in the regions above disclosed, such as, but not limited to the 12 sequences disclosed. The PCR products are obtained and analyzed by any conventional method, such as but not limited to agarose gel electrophoresis, dot-blot hybridizations, Southern blotting, Northern blotting and similar RT-PCR, PCR-ELISA, and others known in the art (for example, but not limited to, Molecular Diagnostic PCR handbook. (2005), Gerrit J. Viljoen, Louis H. and John R. Crowther Nei. Springer Publishers) to correctly identify C. albicans in a multiplex assay or single assay. Note that these oligonucleotides may comprise nucleotide unmarked or marked, such as but not limited to, radioactive labeling, brand quiomiluminiscente, luminescent, fluorescent, biotinylated.

Experimental selected examples, which must be considered only as supporting technical evidence, but without limiting the scope of the invention, are provided herein below.

### EXAMPLES

### Example 1

### Oligonucleotide Design

Candida albicans oligonucleotides and probes were specifically designed from unique sites located on the genome. Non-limiting examples of the specifically designed oligonucleotides are disclosed in Table 1.

TABLE 1
Examples of oligonucleotides for the
identification of Candida albicans.

| Oligonu- | | Forward | | | | |
|---|---|---|---|---|---|---|
| cleotide | | (Fw) | | | Amplicon | |
| pair | Seq. ID. | or Re- | bp | 5′ a 3′ | length | Contig |
| number | No. | verse (Rv) | No. | Sequence | (bp) | Name |
| Ca1 | Seq. ID. | Fw | 18 | AAAGAGCCT | 322 | XM_707573.1 |
| | No. 1 | | | GCGACTATG | | (ALS3-like) |
| | Seq. ID. | Rv | 18 | GGCTGATTC | | |
| | No. 2 | | | TTCGGTTTG | | |
| Ca2 | Seq. ID. | Fw | 20 | CTACTTCCAC | 202 | XM_707573.1 |
| | No. 3 | | | AGCTGCTTCC | | (Chrom R) |
| | Seq. ID. | Rv | 20 | GTTGAAGTTG | | |
| | No. 4 | | | CAGATGGAGC | | |
| Ca3 | Seq. ID. | Fw | 20 | CCTCCTGTAG | 266 | WG_03305 |
| | No. 5 | | | TATTTCTCCC | | (MYO5) |
| | Seq ID. | Rv | 20 | GGAGGGAAGA | | |
| | No. 6 | | | GATGGTAATC | | |
| Ca4 | Seq. ID. | Fw | 20 | CATCAATACC | 221 | WT_03305 |
| | No. 7 | | | ACCCACAAGC | | (MYO5) |
| | Seq. ID. | Rv | 20 | CACCTACTGG | | |
| | No. 8 | | | AATCATCCTC | | |
| Ca5 | Seq. ID. | Fw | 20 | GGTAGCTGGA | 203 | WG_03305 |
| | No. 9 | | | TCTACAGTTC | | (Chrom 4) |
| | Seq. ID. | Rv | 18 | GAACAACGT | | |
| | No. 10 | | | ATCTCCACG | | |
| Ca6 | Seq. ID. | Fw | 18 | CAGTGAACG | 173 | WG_03102 |
| | No. 11 | | | GAAGCTAAG | | (Chrom R) |
| | Seq. ID. | Rv | 21 | CTCTTGATTAA | | |
| | No. 12 | | | CTTGGCCAGG | | |

For example, oligonucleotide pair Ca2 is located in Chromosome R, found in CGD (Candida Genome Database) as: ALS3 orf19.1816 on Ca21 chrR_Ca_SC5314 nt 1535813-1532346. Oligonucleotide Ca5 is located at MYO5 orf19.738 on Ca21 chr4_Ca_SC5314 nt 1096185-1092235. Oligonucleotides pairs Ca3 and Ca4 are located in the intergenic region between hypothetical protein Ca019.740 mRNA and CAWG_{—}03305, protein MYO5 mRNA. Oligonucleotides pair Ca6 is located in Chromosome R at Supercontig 2: 2296345-2296656+Broad Institute MIT Data Candida CAWG_{—}031102.

Said oligonucleotide pairs were tested for optimizing the amplification conditions. Thus, oligonucleotide pairs Ca1 to Ca 6 have annealing temperatures from about 54° C. to 61° C. These oligonucleotide pairs were tested on genomic DNA for amplification testing carrying out PCR reactions. For example the oligonucleotide pairs were analyzed in a final product volume of 30 μL, as follows:

TABLE 2
PCR general experimental conditions.

| Reagents | Concentration | Volume (μL) |
|---|---|---|
| Genomic DNA | Variable | 0.5 μL |
| Buffer 10X | 1X | 3.0 μL |
| MgCl₂ 20X | 1X | 1.5 μL |
| dNTPs 2 Mm | 30 μM | 0.45 μL |
| Primer Forward | 500 nM | 3.0 μL |
| Primer Reverse | 500 nM | 3.0 μL |
| Amplificase | 500U | 0.4 μL |
| Water | | 18.15 μL |
| | Final Volume | 30.0 μL |

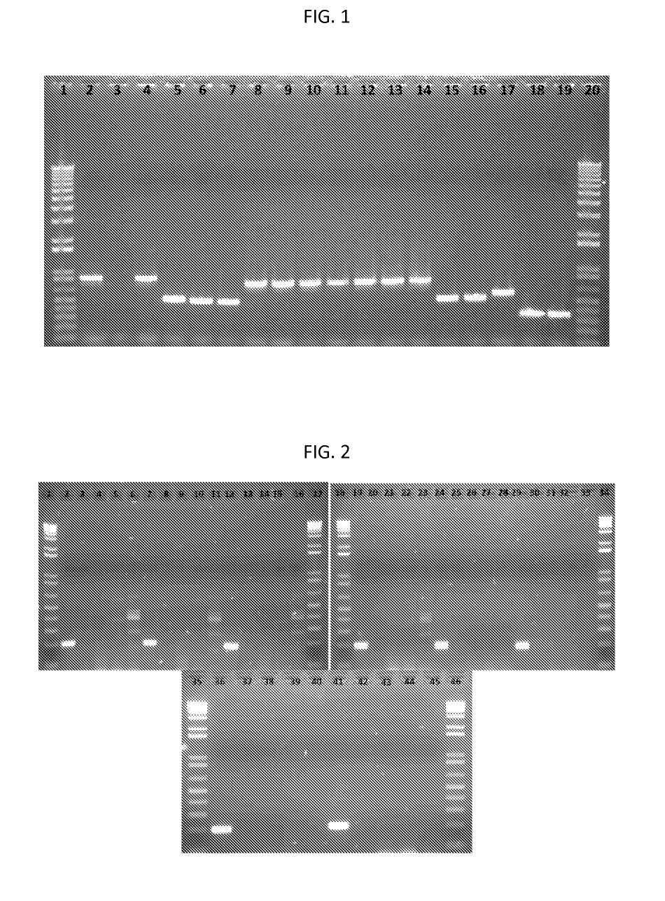

As a control, the quality of the genomic DNA was evaluated by amplifying rDNA regions with universal oligonucleotides ITS1 and ITS4 (Table 3), using the same concentrations and final volume as above disclosed. The genomic DNA was pure, non-degraded and free of molecules that could interfere with further PCR reactions. (FIG. 1)

TABLE 3
Universal oligonucleotides for
amplifying ITS on fungi genes.

| Name | 5′ a 3′ Sequence | Length |
|---|---|---|
| ITS1 | TCCGTAGGTGAACCTGCGG | 19 |
| ITS4 | TCCTCCGCTTATTGATATGC | 20 |

The amplified fragments resulting from the PCR reactions of each oligonucleotide pairs were tested on 2% agarose gels during 60 minutes at 100-130 volts.

During electrophoresis, the samples belonging to other Candida species different to Candida albicans, were loaded at higher concentrations to those used for positive and negative controls. This was made in order to be sure of the oligonucleotide's sensitivity.

### Example 2

### Standardization Techniques

Herein below, standardization results from some selected oligonucleotides are shown. This selection should not be taken as limiting the scope of the invention, but to illustrate the applicability of all the designed oligonucleotides.

3 oligonucleotide pairs are shown in order to reflect the sensitivity and selectivity of the 12 oligonucleotides and probes for identifying C. albicans. These examples are illustrative but not limitative for the scope of the invention.

Optimal PCR reaction conditions: 3 selected oligonucleotide pairs were tested for optimal PCR reaction conditions.

Firstly, annealing conditions were tested with a temperature threshold. Results are shown in Table 4.

Annealing temperatures were tested for each oligonucleotide pairs, the maximum and minimum temperatures wherein the reaction is effective was pointed out in the termocycling and the intermediate temperatures were calculated.

TABLE 4
Annealing temperatures were tested for each oligonucleotide
pairs.

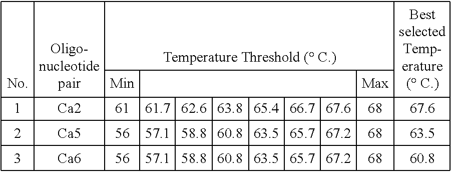

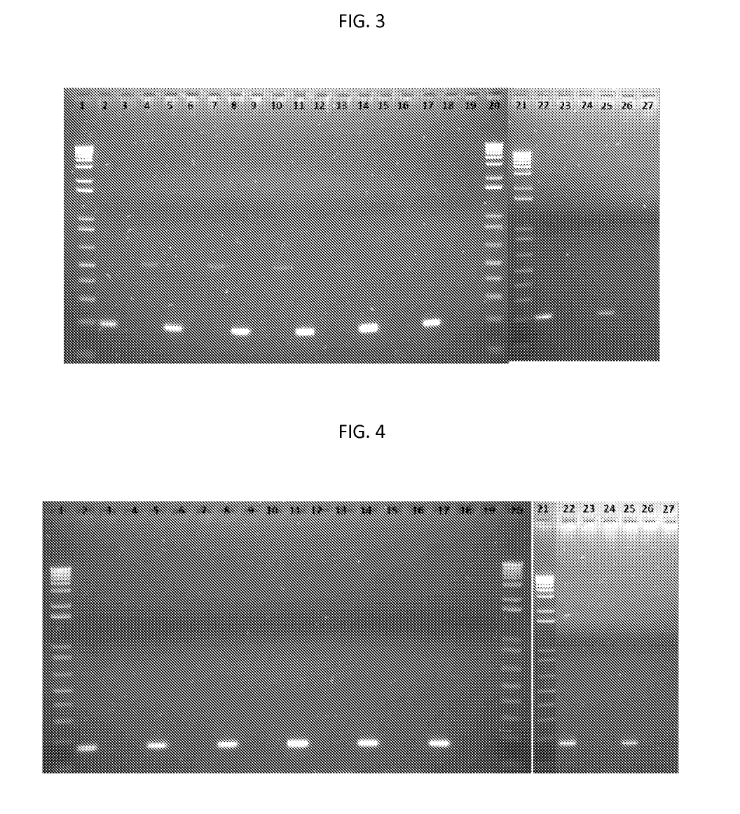

FIGS. 2 to 4 show the minimum temperature threshold wherein the oligonucleotides are more specific compared with other species which show unspecific bands in the first analysis. All the agarose gels are at a concentration of 2% and were run at 110-130 V.

Oligonucleotide Concentration

Once the optimal annealing temperature has been selected for each oligonucleotide pair, the optimal oligonucleotide concentration was determined for PCR reactions.

The concentrations tested were: 100 nM, 200 nM, 400 nM, 500 nM, 600 nM, 800 nM, 1000 nM y 1200 nM.

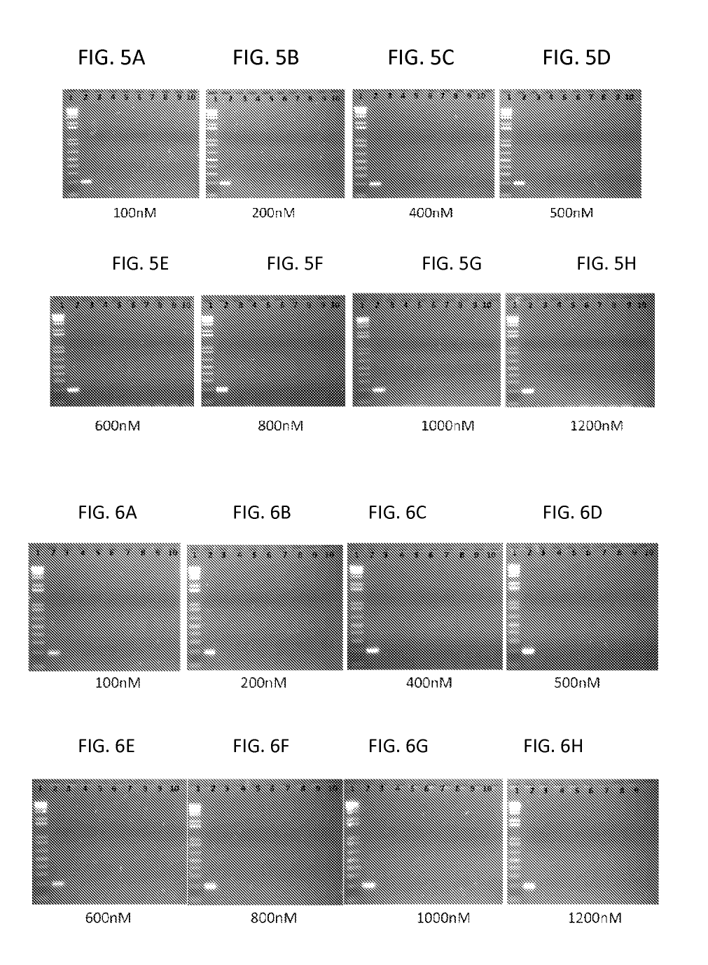

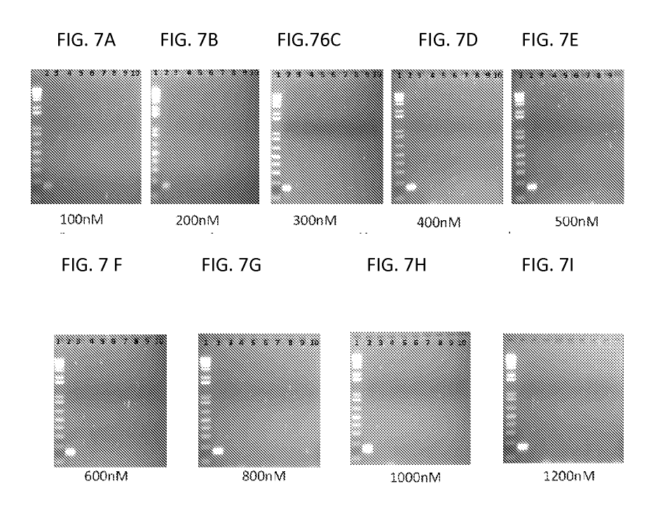

The minimal oligonucleotide concentration wherein a clear band was detected in the positive control, was selected. Table 5 shows the best concentrations. FIGS. 5 to 7 show the optimization results with exemplifying oligonucleotide pairs. All the agarose gels are at a concentration of 2% and were run at 110-130 V.

TABLE 5
Best oligonucleotide concentration for oligonucleotide pairs
designed for C. albicans.

| No | Oligonucleotide pair | Best selected concentration |
|---|---|---|
| 1 | Ca2 | 200 nM |
| 2 | Ca5 | 200 nM |
| 3 | Ca6 | 300 nM |

Genomic DNA Detected.

The amount of genomic DNA that can be detected for each oligonucleotide pair was tested from 100 ng to 0.02 ng with a control without DNA. For C. albicans, genomic DNA can be detected in an amount of at least 1 ng.

### Example 3

### Candida Detection on Isolated Clinical Samples

The above exemplified oligonucleotide pairs were tested to detect Candida albicans on clinical isolated samples from hospitalized patients.

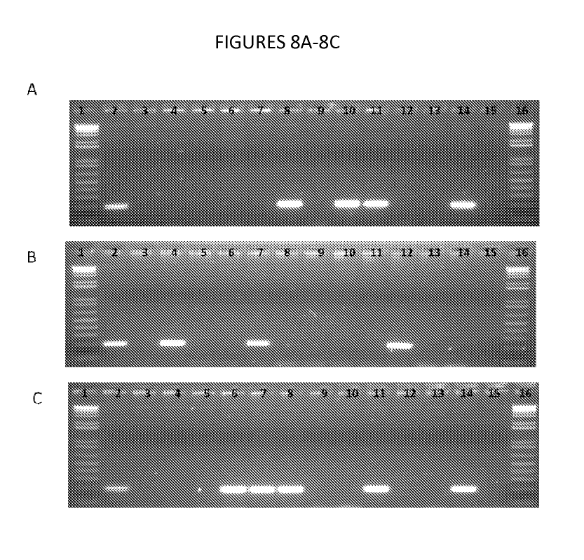

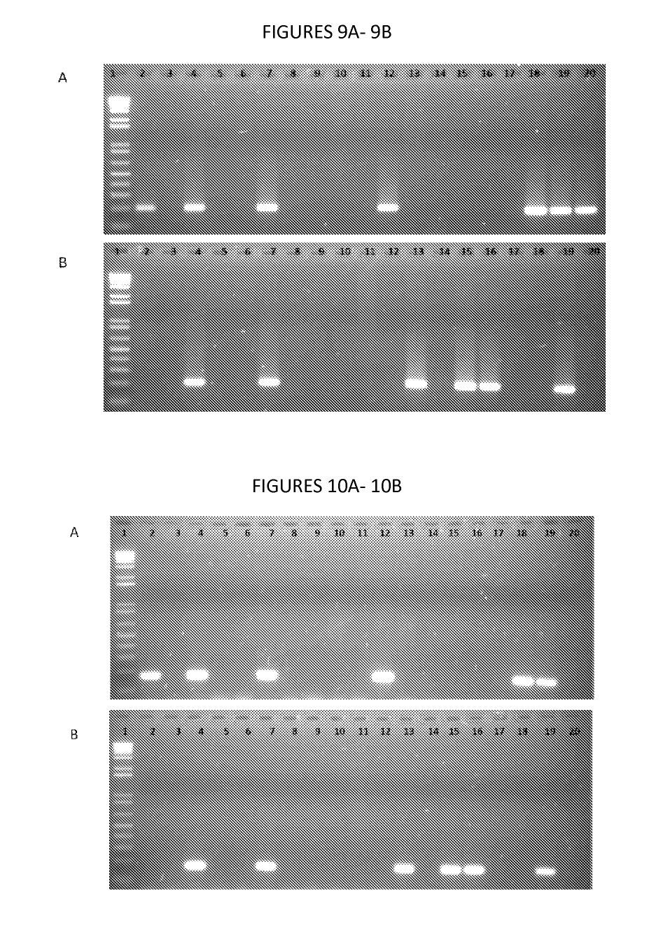

FIGS. 8 to 10 show the results of said tests. All the oligonucleotide pairs detect only the specific Candida specie for which they were designed. In most of the cases all the oligonucleotide pairs detect the same positive samples with two exceptions: Ca6 from C. albicans pair, detect one less sample than the other two pairs from the same specie (Ca2 and Ca5). All the agarose gels are at a concentration of 2% and were run at 110-130 V.

Comparing PCR results with Vitek identification methods reveals that PCR test has a sensibility of 98% and a specificity of 100% in contrast to VITEK tests with an 85% and 33% respectively.

### Example 4

### Multiplex Assay

Since it is possible to have rearrangements within the genome of C. albicans, as shown with clinical sample 17 (see FIG. 10, lane 20) a multiplex assay was designed in order to confirm with 100% specificity, the presence of the microorganism in clinical samples. Since the oligonucleotide pairs are located in several chromosomes, the probability of having more than one rearrangement within a clinical sample is low.

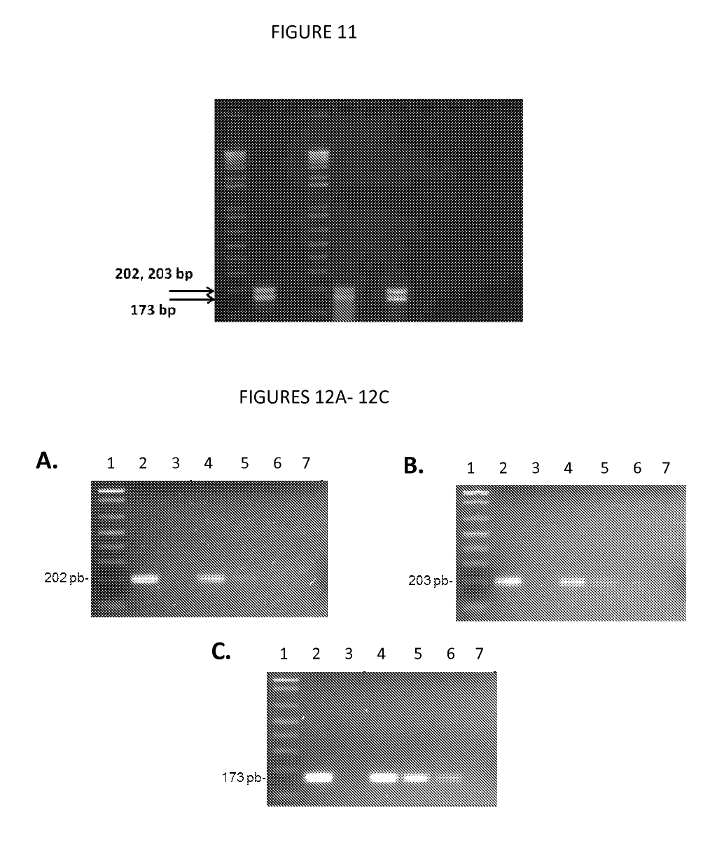

FIG. 11 shows the use of oligonucleotides pairs Ca2, Ca5 and Ca6 simultaneously in samples containing C. albicans alone or in mixture with C. glabrata, C. tropicalis, C. parapsilosis, C. dubliniensis, S. cerevisiae, wherein each microorganism is in an amount of 100 ng. As predicted, the amplified fragments are present only in the lanes containing C. albicans, and not in the control lanes (lane 3, 6, 8 to 11). Therefore, a multiplex kit for detecting C. albicans has been designed with a 100% of sensibility and specificity.

### Example 5

### Specificity Assay

FIG. 12 panels A to C show that the oligonucleotides tested are specific for C. albicans and do not cross-link with other microbial species. For example, C. albicans mixed with another 10 microbial species such as C. tropicalis, C. parapsilosis, C. glabrata, C. dubliniensis, C. bracarensis, C. guilliermondii, C. krusei, C. metapsilosis, C. orthopsilosis, S. cerevisiae (50 ng each for a total of 500 ng). C. albicans DNA was added in different amounts: 100 ng, 10 ng, 1 ng and a control without DNA. As shown, the amplified bands detected correspond to the predicted size (202 bp for Ca2, 203 bp for Ca5 and 173 for Ca6) and its resequencing test. Negative control without C. albicans DNA did not show any amplification band. This confirms that the assay is 100% specific for C. albicans.

Finally, from the totality of clinical samples tested, 12 were classified as C. albicans with a 100% sensitivity and specificity, compared with Vitek tests.

### Example 6

### Real-Time PCR Assay

A positive control was generated by subcloning amplicons derived from the oligonucleotides pairs within a suitable vector, according to manufacturer's instructions. DNA concentration was calculated by 260/280 absorbance readings. Suitable negative controls were also included.

Real-time PCR reactions were performed as follows: Annealing temperature 67° C., oligonucleotide concentration 150 nM (forward and reverse), each point of the standard curve was run in duplicate at 10⁸, 10⁶, 10⁴, 10² dilutions. 40 cycles were run. The linear detection range was from 10⁸ to less than 100 copies per reaction. To confirm amplification quality, the real time PCR products were resequenced and they correspond to the predicted amplicon sequence in at least 5 repeats. Since the samples did not form any primer dimers, this indicates the ability of real-time PCR to efficiently amplify a specific target not only from the positive-control plasmid, but also from more complex DNAs, such as clinical samples. In clinical samples, no significant difference in C_{T} score was apparent (data not shown).

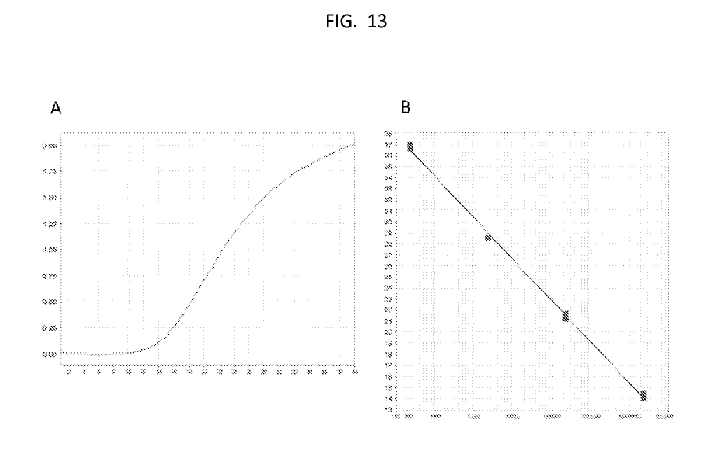

FIG. 13 shows an example of the real-time PCR standard curve using one of the oligonucleotide pairs (Ca2). As shown in panel B, the copy number detected is 85 copies. When resequenced, the amplicon contains the predicted sequence of 202 pb in a 100% match.

What is claimed is:

1. An oligonucleotide for the specific identification of Candida albicans, comprising a nucleic acid having at least 90% sequence homology to one of SEQ ID NOS: 1 to 12 or a complement thereof.

2. An in vitro method for the specific identification of C. albicans, comprising the steps of:
a) amplifying DNA fragments from a biological sample with at least one oligonucleotide as defined in claim 1; and
b) identify the amplified DNA fragments.

3. The method according to claim 2, wherein the amplification of DNA fragments is carried out with at least one pair of oligonucleotides as defined in claim 1.

4. The method according to claim 2, wherein the amplification of DNA fragments is carried out with at least two pair of oligonucleotides as defined in claim 1.

5. A kit for the specific identification of Candida albicans, comprising at least one oligonucleotide as defined in claim 1.

6. The kit according to claim 5, comprising at least one oligonucleotide pair as defined in claim 1.

7. The kit according to claim 6, comprising at least two pair of oligonucleotides as defined in claim 1.
